(19) United States
(12) Patent Application Publication
(10) Pub. No.: US 2018/0050148 A1
(43) Pub. Date: Feb. 22, 2018

(54) METHODS FOR DELIVERING REGIONAL CITRATE ANTICOAGULATION (RCA) DURING EXTRACORPOREAL BLOOD TREATMENTS

(71) Applicant: Aethlon Medical, Inc., San Diego, CA (US)

(72) Inventor: Rodney S. Kenley, Libertyville, IL (US)

(21) Appl. No.: 15/567,500

(22) PCT Filed: Apr. 20, 2016
(86) PCT No.: PCT/US16/28482

§ 371 Date: Oct. 18, 2017

Related U.S. Application Data
(60) Provisional application No. 62/151,934, filed on Apr. 23, 2015.
(60) Provisional application No. 62/210,363, filed on Aug. 26, 2015.

(51) Int. Cl. A61M 1/36; A61M 1/34; A61M 1/16; B01D 61/24; A61K 35/14; A61K 31/194

(52) U.S. Cl. CPC A61M 1/3672; A61M 1/3406; A61M 1/1696; A61M 2205/3317; A61K 35/14; A61K 31/194; B01D 61/243

(57) ABSTRACT

Disclosed are methods, compositions, and devices for improved delivery of regional citrate anticoagulation during extracorporeal blood treatments. Methods comprise quantification of the clearance of calcium and/or citrate using one or more on-line/in-line sensors, establishing a correlation between the differential conductivity between afferent and efferent dialysate and the clearance of calcium and/or citrate. The methods described herein further include quantifying citrate clearance using glucose as a surrogate.

### RELATED APPLICATIONS

This application is an international application that claims the benefit of priority to U.S. Provisional Patent Application Nos. 62/151,934, filed Apr. 23, 2015 and 62/210,363, filed Aug. 26, 2015, the disclosures of which are incorporated by reference herein in their entirety.

### BACKGROUND OF THE INVENTION

### Field of the Invention

The present disclosure relates generally to systems and methods for the prevention of blood clotting during extracorporeal blood circulation therapies. More particularly, the present disclosure relates to systems and methods for delivering regional citrate anticoagulation (RCA) during extracorporeal blood treatments.

### Description of the Related Art

Although regional citrate anticoagulation (RCA) has been used for decades in many extracorporeal blood circulation therapies and is well known for the prevention of clotting in extracorporeal circuits containing blood treatment devices for 24 hours and longer, its implementation is complex and fraught with complicated formulation of custom dialysates. Furthermore, the delivery of RCA requires frequent monitoring of the patient's electrolyte and acid/base status. A variety of different protocols have been developed and promoted by various investigators but, to date, no standardization around a single protocol has evolved. Thus, RCA delivery continues to be a highly manually-intensive and monitoring-intensive anticoagulation technique. Furthermore, no instrumentation has ever been developed or submitted to the FDA that purports to control and automate the process so as to reduce to a minimum, the required degree of human intervention and increase to a maximum, the safety inherent in using such a system.

Fundamentally, RCA is accomplished by infusing a citrate-containing solution into the arterial limb of an extracorporeal circuit as close as possible to the blood access device to insure anticoagulation of the largest possible length of the circuit. The addition of trivalent citrate anions has the effect of chelating both ionized calcium and magnesium divalent cations. By infusing sufficient citrate to reduce the ionized calcium concentration to below 0.35 mmol/L in the incoming blood, coagulation, which is dependent on ionized calcium, is prevented. In some extracorporeal procedures such as blood collection/cell separation, the citrate/calcium complexes are simply returned to the donor.

Allowing the calcium-citrate complexes to return to a healthy patient during a 1-2 hour blood donation event does not elicit any clinical sequelae because it is easily metabolized to bicarbonate and CO₂ by the mitochondria of the liver, muscle and skeletal tissue. However, returning the citrate/calcium to a patient undergoing continuous renal replacement therapy (CRRT) for acute kidney failure or one who is trending towards sepsis with a possible hyporesponsive liver must be prevented. Otherwise, the patients can develop citrate toxicity, hyper or hypocalcemia, acid/base derangements, and a host of other sequelae such as arrhythmias which can be fatal.

Currently, this is typically avoided by allowing the calcium/citrate complexes to diffuse through a dialyzer or hemofilter membrane which is being perfused by dialysate or by extracting them convectively with copious amounts of ultrafiltration in straight hemofiltration procedures or both in the case of hemodiafiltration. When using these procedures, the calcium lost to the effluent must be replaced into the patient's blood in order to avoid hypocalcemia. This is typically accomplished by infusing a solution of calcium chloride or calcium gluconate into the venous return limb of the extracorporeal circuit, again, as close to the blood access connection as possible so as to bring the patient's ionized calcium concentration back into the 0.9-1.3 mmol/L physiologic range.

### SUMMARY OF THE INVENTION

One of the principal factors contributing to the difficulty in providing citrate anticoagulation is the fact that determining the correct rate at which to infuse calcium into the venous blood line is dependent on knowing how much citrate/calcium has been cleared from the circuit. This, in turn, is dependent on the blood flow rate, the dialysate flow rate, the ultrafiltration rate, and the surface area of available membrane in the dialyzer/hemofilter; all of which can change intra-treatment. It is also important to know the amount of citrate being removed from the circuit because this translates into the remaining quantity being delivered to the patient, which must be compensated for by adjusting the amount of bicarbonate in the dialysate or replacement fluid in order to keep the patient in a correct acid/base balance.

The impediment to automating this process to this point has been the absence of knowledge of the exact amount of calcium and citrate being cleared from the circuit. In order to pass regulatory safety muster, one must be able to assure at any point in time that the composition of blood returning to the patient is nearly identical to that which came from the patient (within reasonable clinical margins). Since the performance of a dialyzer/hemofilter cannot be assured to remain constant over the course of a 24-hour or longer treatment, being able to quantify the citrate and/or calcium being transported through the membrane and into the effluent dialysate becomes mandatory if the objective is to claim automation and safety.

Automating and standardizing this process could be accomplished with the development of on-line/in-line sensors, preferably located in the effluent dialysate circuit, which would allow the quantification of the clearance of calcium and citrate. Accordingly, in one embodiment described herein is a method for establishing a correlation between the differential conductivity between afferent and efferent dialysate and the clearance of both calcium and citrate.

One of the options for a citrate infusate is tri-sodium citrate. Once citrate from this solution chelates calcium or magnesium in the blood to which it is infused, sodium ions are liberated. These sodium ions will be cleared at a rapid rate when a diffusive modality such as dialysis or hemodiafiltration is employed, which will increase the conductivity of the dialysate into which it is dispersed. It is reasonable to expect that this conductivity could be sensed as it enters the effluent dialysate upon passage through the dialyzer/hemofilter membrane and could, in turn, be correlated with the actual citrate/calcium clearance separately measured during development by a gold standard instrument. If the correlation is repeatable over a range of flow rates and dialysate compositions, the conductivity differential could then be used as an accurate surrogate of citrate and/or calcium clearance. Further accuracy can be accomplished if both afferent and efferent dialysate streams were passed through the same conductivity sensor thereby eliminating any variations between two independent sensors.

In some embodiments, systems and methods are provided which allows for regional citrate anticoagulation in an extracorporeal circuitry, wherein the system comprises an extracorporeal circuitry comprising a dialysate circuit passing through a hemofilter, one or more sensors for detecting the differential conductivity between afferent and efferent dialysate, and automation hardware and software that calculates the clearance of calcium and citrate and automates the reinfusion of ionized calcium into the venous return leg of the extracorporeal blood path closest to the patient. In some embodiments, the method further comprises infusing the blood with citrate into the arterial limb of an extracorporeal circuit as close as possible to the blood access to reduce the ionized calcium concentration, and returning the blood to the subject with physiological levels of calcium. In some embodiments, the citrate is tri-sodium citrate, wherein citrate chelates calcium, thereby forming a calcium/citrate complex. In some embodiments, the level of ionized calcium is decreased to less than 0.35 mmol/L but, in some embodiments, the level of ionized calcium is greater than zero. In some embodiments, calcium is infused back into the blood just prior to returning the blood to the subject, wherein the concentration of the ionized calcium is restored to physiological levels of 0.9-1.3 mmol/L (e.g., 0.9, 1.0, 1.1, 1.2, or 1.3 mmol/L or within a range defined by any two of the aforementioned concentrations).

In some embodiments, a method is provided which allows for regional citrate anticoagulation, wherein the method comprises introducing blood into an extracorporeal system comprising a blood path and a dialysate path on opposite sides of a semipermeable membrane contained in a hemofilter, an affinity cartridge, and a fluorometer. In some embodiments, the affinity cartridge comprises a lectin (e.g., Concanavalin A, Galanthus nivalis lectin (GNA), Lens culinaris (LCH), Ricinus communis Agglutinin (RCA), Arachis hypogaea (PNA), Artocarpus integrifolia (AIL), Vicia villosa (VVL), Triticum vulgaris (WGA), Sambucus nigra (SNA), Maackia amurensis (MAL), Maackia amurensis (MAH), Ulex europaeus (UEH), or Aleuria aurantia (AAL) or any combination of lectins thereof). In some embodiments, a fluorescently labeled dextran is bound to the lectin. In some embodiments, the fluorescently labeled dextran is labeled with fluorescein isothiocyanate (FITC). In some embodiments, the method comprises diffusing glucose from the blood into the effluent dialysate flow path during blood recirculation. In some embodiments, the glucose passes through the lectin affinity cartridge binding to the lectin contained therein and displacing the fluorescently labeled dextran, whose concentration and rate of displacement is quantified by a downstream fluorometer and correlated to the clearance rate of glucose from blood which, in turn, is correlated to the clearance rate of citrate and calcium from blood. In some embodiments, the quantity of glucose levels is used in a feedback loop to determine the infusion rate of calcium into the venous blood path.

In some embodiments, a method for providing regional citrate anticoagulation is provided, wherein the method comprises introducing blood into an extracorporeal system comprising a selective cytopheretic device (SCD), an anion exchange cartridge, a hemofilter, and one or more sensors. In some embodiments, citrate is introduced into the system, and chelates calcium, forming a calcium/citrate complex. When blood or dialysate containing these calcium/citrate complexes pass over certain anion exchange resins, the citrate will be preferentially bound to the resin and exchanged for chloride ions. This has the effect of liberating the calcium ions that were previously bound to the citrate resulting in the composition of the solution exiting the anion exchange cartridge being mostly devoid of calcium citrate and instead, mostly populated by calcium chloride. In some embodiments, the method comprises returning the calcium that was previously extracted from the blood by citrate chelation to the patient by liberating it from the citrate via its passage over an anion exchange resin after it has passed through the bulk of the extracorporeal circuit including any blood treatment devices contained therein.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments, and are not intended to be limiting in scope.

FIG. 1 is a schematic diagram of one embodiment of a method for citrate clearance determination, wherein both afferent and efferent dialysate streams are passed through the same conductivity sensor thereby eliminating any variations between two independent sensors.

FIG. 2 is a schematic diagram of one embodiment of the method for citrate clearance determination, depicting the use of glucose as a surrogate for citrate. The glucose diffuses from the blood of a patient through the semipermeable membrane of a hemofilter into the effluent dialysate flow path and is introduced to an affinity cartridge having a lectin bound thereto. Fluorescently labeled dextran is bound to the lectin. Fluorescently labeled dextran is displaced by the glucose, and the concentration of displaced labeled dextran is detected and quantified. A high degree of correlation between the displacement of glucose and the clearance of citrate and calcium allows the fluorometer reading to be translated into calcium and citrate clearance values. These values are then used in a feedback loop to set the infusion rate of calcium into the venous blood such that the ionized calcium concentration of the returning blood is at or close to the prescribed value.

FIG. 3 is a schematic diagram of one embodiment of a method of providing RCA when a selective cytopheretic device (SCD) and a downstream hemofilter are located in the blood path and the dialysate is recirculated through both devices and an anion exchange cartridge from a single reservoir. This embodiment illustrates how the dialysate, containing a large majority of calcium/citrate complexes that have diffused into it from the blood during its transit through the hemofilter can be recirculated through an anion exchange cartridge where the citrate is bound and exchanged for chloride thereby liberating the calcium ions previously removed from the patient's blood and directing this calcium chloride-containing dialysate back through the hemofilter where the calcium will diffuse back into the calcium-poor blood just prior to returning to the patient.

FIG. 4 is a schematic diagram of an embodiment similar to that depicted in FIG. 3 except that the dialysate is sent from a reservoir to a drain in a single pass format rather than recirculating it.

FIG. 5 is a schematic diagram of one embodiment wherein citrate capture and calcium release/reinfusion is implemented using an anion exchange cartridge in conventional modes of intermittent or continuous renal replacement therapy where no SCD in employed.

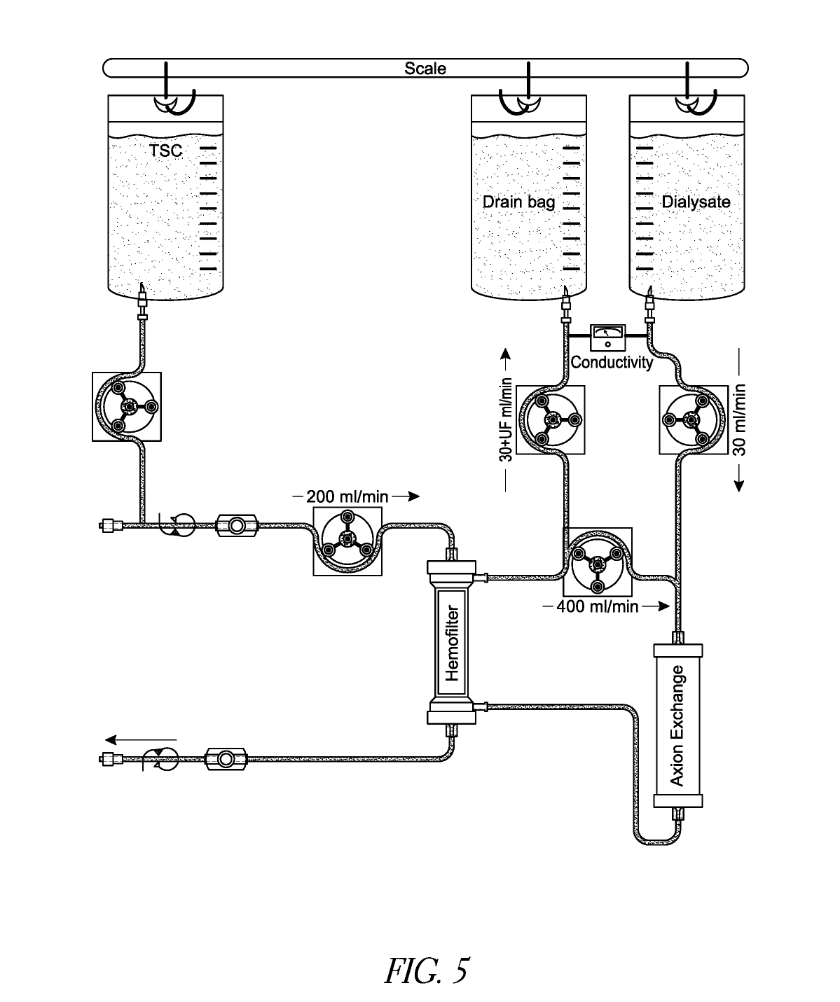

### DETAILED DESCRIPTION OF THE PREFERRED EMBODIMENTS

Although the invention is described in various exemplary embodiments and implementations as provided herein, it should be understood that the various features, aspects, and functionality described in one or more of the individual embodiments are not limited in their applicability to the particular embodiment with which they are described. Instead, they can be applied alone or in various combinations to one or more of the other embodiments of the invention, whether the embodiments are described or whether the features are presented as being a part of the described embodiment. The breadth and scope of the present invention should not be limited by any exemplary embodiments described or shown herein.

As used herein, the term “glucose exchange medium” refers to a medium which includes, but is not limited to, a resin, a bead, a column, a cartridge, a porous membrane, or other medium through which a solution can pass, and which binds to or is capable of binding to glucose.

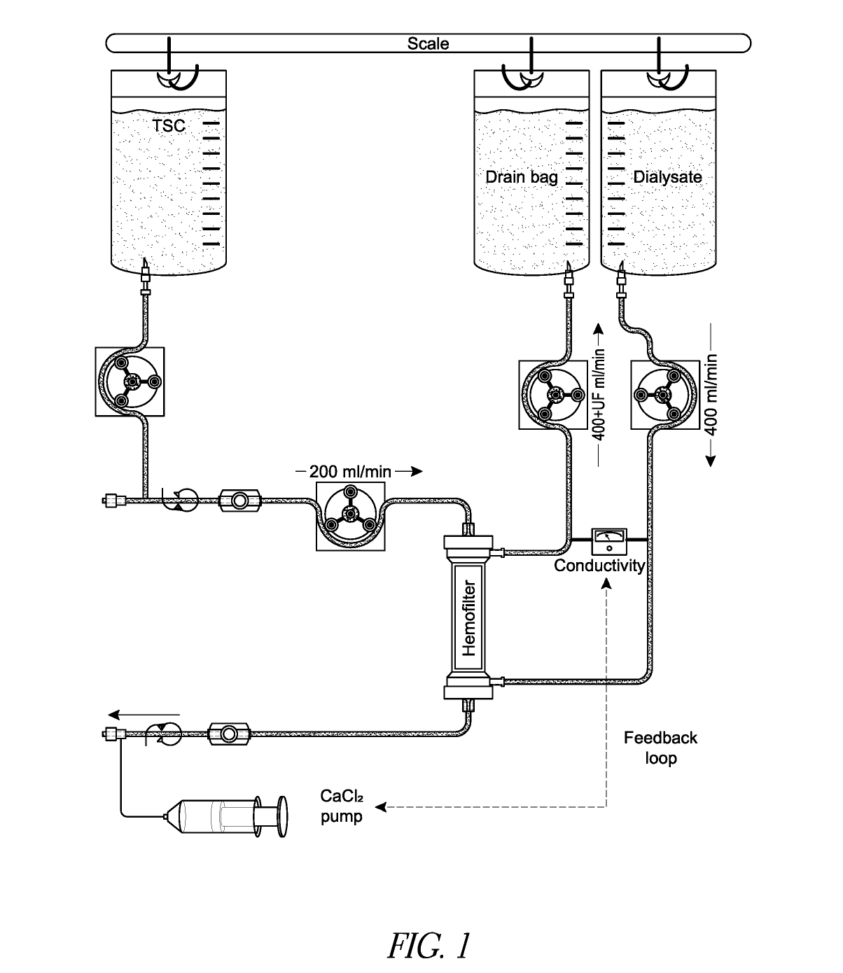

In some embodiments a method is provided for delivering regional citrate anticoagulation to a subject, as depicted in FIG. 1. In this embodiment, the method comprises introducing blood into an extracorporeal system comprising a hemofilter, infusing the blood with citrate to form complexes of citrate/calcium to prevent blood from coagulating in the hemofilter and the blood circuit, flowing the blood through the hemofilter, flowing dialysate through the hemofilter in a flow direction opposite of the blood flow, measuring the conductivity of the dialysate prior to passage through the hemofilter and measuring the conductivity of the dialysate after passage through the hemofilter to determine a differential conductivity, infusing blood that has passed through the hemofilter with calcium chloride based on the measured differential conductivity, and returning the blood to the subject. In some embodiments the method comprises monitoring the differential conductivity between the afferent and the efferent streams of dialysate, determining an actual clearance of calcium and of citrate through the membrane, and correlating the differential conductivity to the actual clearance of calcium and citrate. In some embodiments, the citrate is infused into the blood immediately upon entering the extracorporeal system in order to ensure that the blood does not coagulate. In some embodiments, the flow rate of the blood through the extracorporeal system is about 200 mL/min. One of skill in the art would recognize that the flow rates may be adjusted based on the specific application, capabilities, or needs of the method. In some embodiments, the flow rate of the dialysate is at least twice that of the flow rate of the blood, for example, at least 400 mL/min.

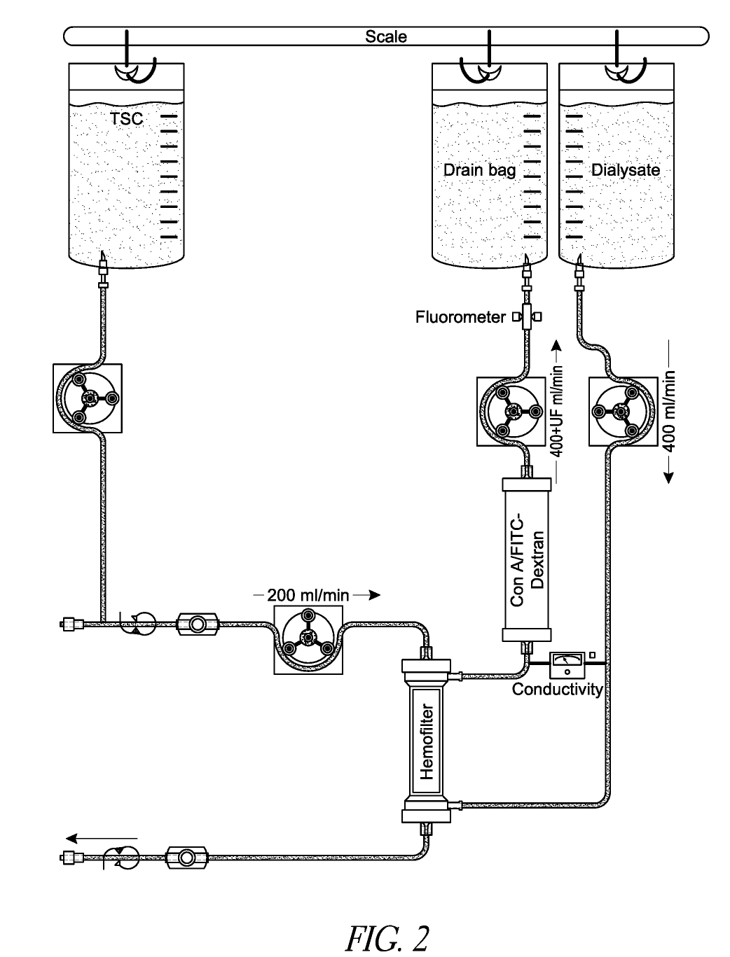

In some embodiments a method is provided for quantifying citrate clearance in an extracorporeal system using glucose as a surrogate, as depicted in FIG. 2. This approach takes advantage of the fact that glucose and citrate are nearly identical in molecular weight and therefore their transport through a dialysis membrane is very similar. In this case, the preferred citrate infusate would be that which is most commonly used in RCA: anticoagulant citrate-dextrose-acid or ACD-A as it is commonly known. This solution contains 124 mmol/L of glucose. In some embodiments, the extracorporeal system comprises a lectin affinity cartridge, preferably a lectin affinity cartridge comprising Concanavalin A, which is a lectin that has been well characterized as a strong binder of both monomeric glucose and the glucose polymer, Dextran. In some embodiments, however, the lectin affinity cartridge comprises at least one or more of the following lectins Concanavalin A, Galanthus nivalis lectin (GNA), Lens culinaris (LCH), Ricinus communis Agglutinin (RCA), Arachis hypogaea (PNA), Artocarpus integrifolia (AIL), Vicia villosa (VVL), Triticum vulgaris (WGA), Sambucus nigra (SNA), Maackia amurensis (MAL), Maackia amurensis (MAH), Ulex europaeus (UEH), or Aleuria aurantia (AAL) or any combination of lectins thereof). That is, in some embodiments, in addition to Concanavilin A, the lectin cartridge may contain one or more additional lectins so as to utilize a mixed lectin bed (e.g., one or more lectins selected from the group consisting of Galanthus nivalis lectin (GNA), Lens culinaris (LCH), Ricinus communis Agglutinin (RCA), Arachis hypogaea (PNA), Artocarpus integrifolia (AIL), Vicia villosa (VVL), Triticum vulgaris (WGA), Sambucus nigra (SNA), Maackia amurensis (MAL), Maackia amurensis (MAH), Ulex europaeus (UEH), and Aleuria aurantia (AAL) or any combination of lectins thereof).

In one embodiment, Concanavalin A (Con A), which has previously had Dextran that is labeled with the fluorescent marker FITC bound to it, is sequestered in a flow-through container, which is located in the effluent dialysate flow path. When a glucose-containing solution is passed over this Con A-FITC-Dextran compound, the FITC-Dextran is displaced by the glucose and the intensity of the resultant fluorescence in the effluent fluid can be quantified fluorometrically and correlated to the concentration of glucose in the perfused fluid. Although this technology has been attempted for use in blood glucose sensing particularly for monitoring blood sugar levels in diabetes, it has never been proposed or suggested for use in an extracorporeal blood purification modality. Its application in blood has been problematic due to the fact that Con A is toxic if allowed to be released into a patient's bloodstream. In the proposed application, such a danger is non-existent since the Con A will only be deployed in the effluent dialysate.

If a glucose-free dialysate is used, then glucose in the blood will represent the only source of glucose entering the effluent dialysate. The fluorescence resulting from displacement by glucose molecules could be detected by a non-invasive downstream fluorometer and compared to the actual clearance of citrate and calcium as measured by independent means. If there is a high degree of correlation, then the fluorometer reading can be translated into calcium and citrate clearance values. These values could then be used in a feedback loop to set the infusion rate of calcium into the venous blood such that the ionized calcium concentration of the returning blood is at or near the desired value.

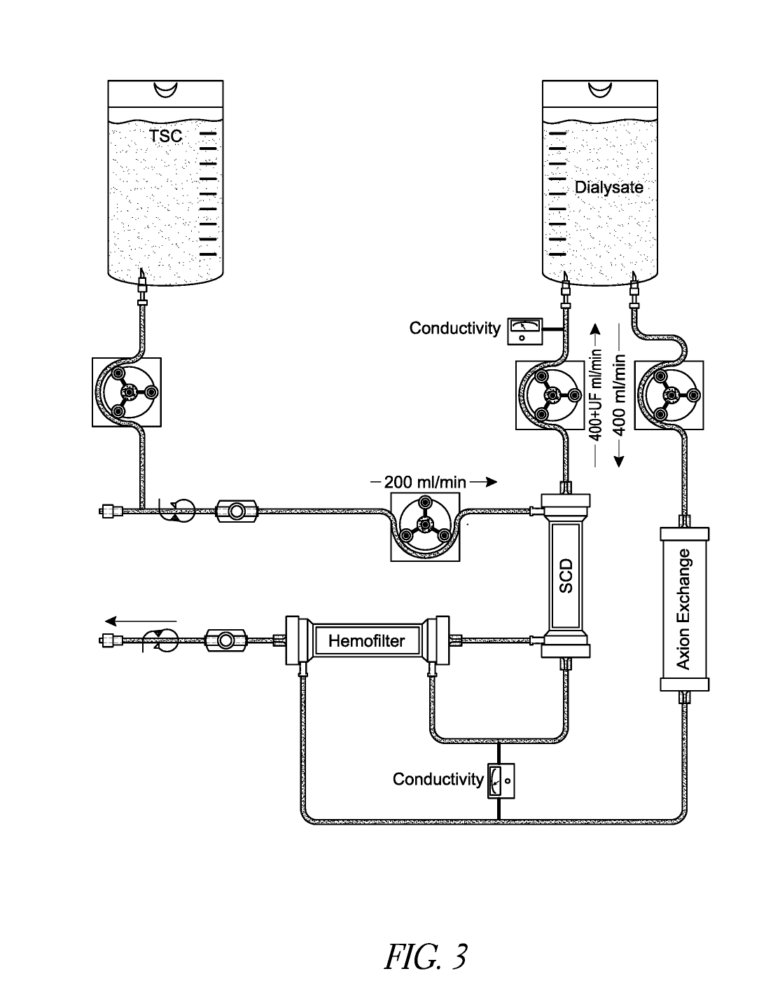

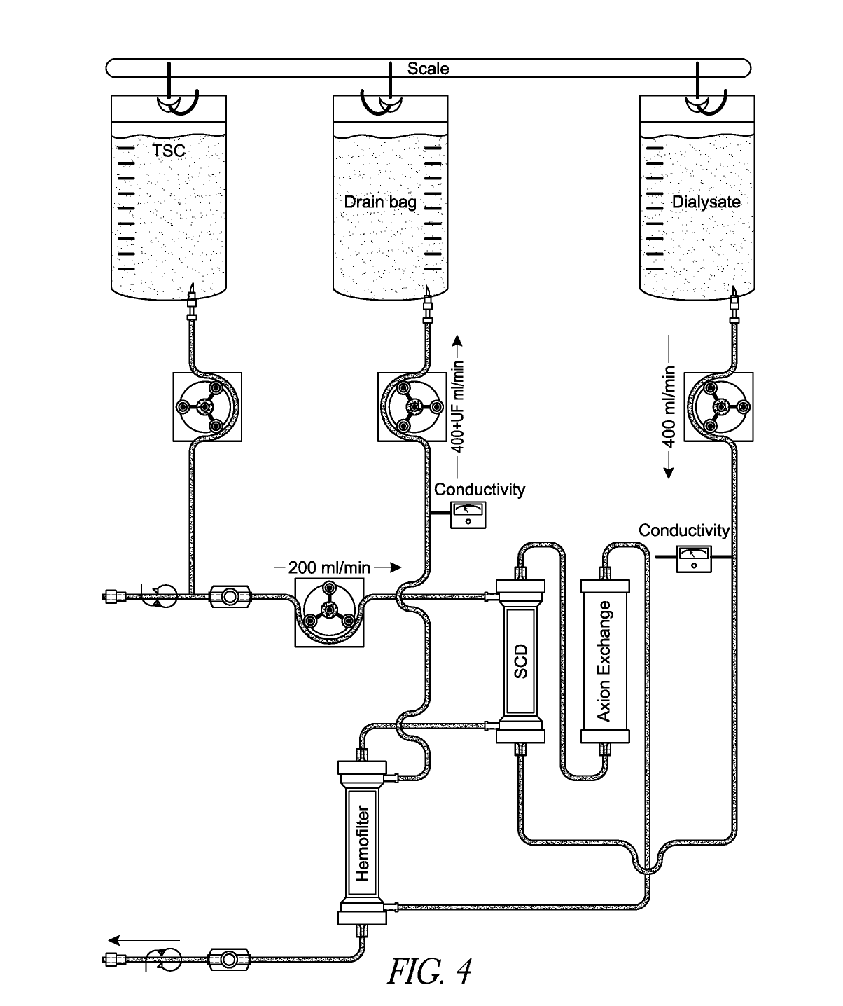

In some embodiments a method is provided for providing regional citrate anticoagulation when a selective cytopheretic device (SCD) is employed in the extracorporeal circuit, as depicted in FIGS. 3 and 4. A conundrum in the provision of citrate anticoagulation to extracorporeal therapies occurs where there is no need for diffusive or convective removal of impurities from the blood (and hence no dialysate) but rather the therapeutic effect is achieved by simply bringing blood into contact with beads or membranes incorporated within the blood treatment device. In these cases, there is no mechanism for extracting citrate from the blood and, as such, typical citrate anticoagulation would not be feasible. Four examples of such devices are the Cytosorb® manufactured by Cytosorbents Corporation, the Hemopurifier manufactured by Aethlon Medical Inc., the SCD developed by Cytopherx, Inc. of Ann Arbor Mich. and the Toraymyxin column distributed by Spectral Diagnostics, Inc.

The case of the Selective Cytopheretic Device (SCD) is of special interest given that its clinical efficacy is dependent on a low ionized calcium environment. The intended use of this device is for treating a variety of inflammation-mediated disease states including sepsis and acute kidney injury. The device is a conventional hollow fiber hemofilter but one where the inner lumens of the hollow fibers are not intended to be perfused but rather whole blood is perfused on the outside of the fibers where dialysate is normally circulated. The company has determined that leukocytes can be largely deactivated by incurring a residence time in the spongy architecture of the outer walls of these fibers, which contributes to an amelioration of the progression of inflammation. However, this deactivation only occurs in a low ionized calcium environment such as that created by RCA.

As such, in some embodiments is a method of using RCA with this device but without requiring the use of large and costly amounts of dialysate whose only purpose when using this device in cases not requiring renal replacement therapy would be to clear citrate so as to avoid its accumulation in the patient. One approach would be to provide a mechanism for extracting the large majority of calcium/citrate complexes formed by the infusion of citrate followed by separating the calcium from the citrate, sequestering the citrate from returning to the blood while reinfusing the calcium previously removed back into the venous blood returning to the patient.

In some embodiments is provided a method for delivering regional citrate anticoagulation including an anion exchange cartridge located in a loop of recirculating dialysate that perfuses the inner lumens of the hollow fiber bundle of the SCD and the outer lumens of the hollow fibers of the hemofilter located downstream of the SCD. If the appropriate anion exchange resin is employed (e.g. AMBERLITE™ FPA90Cl, AMBERLITE™ FPA98Cl, or AMBERLITE™ FPA40Cl), the calcium citrate entering the recirculating dialysate from the blood by diffusion (which can be maximized by running the dialysate flow rate at least twice the blood flow rate) will be bound by the anion exchange resin in exchange for chloride ions and the calcium bound to the citrate will be liberated into the dialysate. As the dialysate is then circulated through a hemofilter or dialyzer downstream of the SCD and near the connection to the patient's blood access device, the same calcium previously extracted from the arterial blood line can be returned to the venous line via diffusion through the hemofilter/dialyzer while the SCD and hemofilter/dialyzer remain anticoagulated.

If the process is 100% efficient or nearly so, then no exogenous infusion of calcium will be necessary and no sensors aimed at quantifying the extraction of citrate and calcium will be necessary. If the process is not sufficiently efficient, then sensors, such as those described above, can be implemented with the aforementioned feedback loop to control the rate of calcium infusion but with significant reductions in the amount of dialysate and calcium infusate required.

This same approach could be used when the SCD is used in conjunction with renal replacement therapy by, instead of recirculating dialysate in and out of the same reservoir, delivering it in a single pass manner as is conventionally done.

In some embodiments a method is provided for delivering regional citrate anticoagulation, wherein the method comprises introducing blood into an extracorporeal system comprising a hemofilter, infusing the blood with citrate to form complexes of citrate-calcium, flowing the blood through the hemofilter, flowing dialysate over an anion exchange cartridge to liberate chloride in exchange for the citrate anions, thereby liberating calcium ions previously complexed with the citrate anions, flowing the dialysate that passed through the anion exchange cartridge through the hemofilter in a flow direction opposite of the blood flow, and returning the blood that passed through the hemofilter to the subject. In some embodiments, the anion exchange cartridge comprises an anion exchange resin selected from the group consisting of AMBERLITE™ FPA90Cl, AMBERLITE™ FPA98Cl, and AMBERLIIE™ FPA40Cl. In some embodiments, the subject is undergoing continuous renal replacement therapy or intermittent dialysis. In some embodiments, the capture and infusion of calcium is accomplished in a dialysate single pass format or in a dialysate recirculation format. In some embodiments, the dialysate flow rate is at least twice that of the blood flow rate.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A method for delivering regional citrate anticoagulation to a subject, comprising:
introducing blood into an extracorporeal system comprising a hemofilter;
infusing the blood with citrate to form complexes of citrate/calcium;
flowing the blood through the hemofilter;
flowing dialysate through the hemofilter in a flow direction opposite of the blood flow;
measuring the conductivity of the dialysate prior to passage through the hemofilter and measuring the conductivity of the dialysate after passage through the hemofilter to determine a differential conductivity;
determining an actual clearance of calcium and of citrate through the membrane;
correlating the differential conductivity to the actual clearance of calcium and citrate;
infusing blood that has passed through the hemofilter with calcium chloride based on the measured differential conductivity; and
returning the blood to the subject.

2. The method of claim 1, wherein the dialysate flow rate is at least twice that of the blood flow rate.

3. The method of claim 2, wherein the dialysate flow rate is 400 mL/min.

4. The method of claim 1, wherein the subject is undergoing continuous renal replacement therapy or intermittent dialysis.

5. The method of claim 1, wherein the capture and infusion of calcium is accomplished in a dialysate single pass format or in a dialysate recirculation format.

6. A method for delivering regional citrate anticoagulation to a subject, comprising:
introducing blood into an extracorporeal system comprising a hemofilter and an effluent dialysate flow path having a glucose exchange medium located therein, and wherein the glucose exchange medium comprises a lectin attached thereto, and is saturated with fluorescently-labeled dextran;
infusing the blood with citrate to form complexes of citrate/calcium;
flowing the blood through the hemofilter;
flowing dialysate through the hemofilter in a flow direction opposite of the blood flow;
flowing the dialysate that passed through the hemofilter through the glucose exchange medium, thereby displacing the fluorescently-labeled dextran;
detecting the displaced fluorescently labeled dextran to determine the concentration of glucose in the effluent dialysate flow path;
infusing the blood that passed through the hemofilter with calcium chloride based on the measured concentration of glucose; and
returning the blood to the subject.

7. The method of claim 6, wherein the porous membrane comprises one or more of the lectins Concanavalin A, Galanthus nivalis lectin (GNA), Lens culinaris (LCH), Ricinus communis Agglutinin (RCA), Arachis hypogaea (PNA), Artocarpus integrifolia (AIL), Vicia villosa (VVL), Triticum vulgaris (WGA), Sambucus nigra (SNA), Maackia amurensis (MAL), Maackia amurensis (MAH), Ulex europaeus (UEH), or Aleuria aurantia (AAL), preferably at least Concanavalin A.

8. The method of claim 6, wherein the fluorescently labeled dextran is labeled with fluorescein isothiocyanate (FITC).

9. The method of claim 6, wherein the glucose exchange medium is selected from the group consisting of a cartridge, a column, a resin, a bead, or a porous membrane.

10. The method of claim 6, wherein the dialysate flow rate is at least twice that of the blood flow rate.

11. The method of claim 6, wherein the subject is undergoing continuous renal replacement therapy or intermittent dialysis.

12. The method of claim 6, wherein the capture and infusion of calcium is accomplished in a dialysate single pass format or in a dialysate recirculation format.

13. A method for providing regional citrate anticoagulation to a subject, the method comprising:
introducing blood into an extracorporeal system comprising a selective cytopheretic device (SCD) and a hemofilter;
infusing the blood with citrate, wherein the citrate binds to calcium in the blood forming complexes of calcium/citrate;
flowing dialysate over an anion exchange cartridge to liberate chloride in exchange for the citrate anions, thereby liberating calcium ions previously complexed with citrate; and
infusing the blood that passed through the hemofilter with calcium chloride; and
returning the blood to the subject.

14. The method of claim 13, wherein the dialysate containing the calcium chloride liberated from the anion exchange resin is passed through the hemofilter in a direction opposite to that of the blood such that the calcium chloride diffuses into the blood and returns to the subject.

15. The method of claim 13, wherein the anion exchange cartridge comprises an anion exchange resin selected from the group consisting of AMBERLITE™ FPA90Cl, AMBERLITE™ FPA98Cl, and AMBERLITE™ FPA40Cl.

16. The method of claim 13, wherein the extracorporeal system further comprises one or more sensors for measuring the conductivity of the dialysate for determination of the amount of calcium chloride to infuse into the blood.

17. The method of claim 13, wherein the subject is undergoing continuous renal replacement therapy or intermittent dialysis.

18. The method of claim 13, wherein the capture and infusion of calcium is accomplished in a dialysate single pass format or in a dialysate circulation format.

19. A method for delivering regional citrate anticoagulation to a subject, comprising:
introducing blood into an extracorporeal system comprising a hemofilter;
infusing the blood with citrate to form complexes of citrate-calcium;
flowing the blood through the hemofilter;
flowing dialysate over an anion exchange cartridge to liberate chloride in exchange for the citrate anions, thereby liberating calcium ions previously complexed with the citrate anions;
flowing the dialysate that passed through the anion exchange cartridge through the hemofilter in a flow direction opposite of the blood flow; and
returning the blood that passed through the hemofilter to the subject.

20. The method of claim 19, wherein the anion exchange cartridge comprises an anion exchange resin selected from the group consisting of AMBERLITE™ FPA90Cl, AMBERLITE™ FPA98Cl, and AMBERLITE™ FPA40Cl.

21. The method of claim 19 or 20, wherein the subject is undergoing continuous renal replacement therapy or intermittent dialysis.

22. The method of claim 19, wherein the capture and infusion of calcium is accomplished in a dialysate single pass format or in a dialysate recirculation format.

23. The method of claim 19, wherein the dialysate flow rate is at least twice that of the blood flow rate.
